(19) United States
(12) Patent Application Publication
(10) Pub. No.: US 2010/0036198 A1
(43) Pub. Date: Feb. 11, 2010

(54) DEVICE FOR THE MANIPULATION OF BODY TISSUE

(76) Inventors: Roberto Tacchino, Roma (IT); Alessandro Pastorelli, Roma (IT); Michele D'Arcangelo, Roma (IT); Federico Bilotti, Aprillia (IT); Jesse J. Kuhns, Cincinnati, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/279,547

(22) PCT Filed: Feb. 12, 2007
(86) PCT No.: PCT/EP2007/001168

§ 371 Date: Jun. 26, 2009

(30) Foreign Application Priority Data
Mar. 13, 2006 (IT) MI2006A000443

(51) Int. Cl. A61B 1/018; A61B 1/313

(52) U.S. Cl. 600/106

(57) ABSTRACT

A device for manipulating body tissue comprises a base structure and at least one tubular member having a proximal end and an orientable distal end that defines a seat for supporting a surgical instrument, in which the tubular member is connected to the base structure. An actuation mechanism is suitable for orientating the distal end of the tubular member to take it into certain operative configurations. The actuation mechanism is also connected to the base structure. Means are also foreseen for the connection of the base structure to a distal end portion of an insertion tube of an endoscope or laparoscope, so that the actuation mechanism and the tubular member are arranged outside the insertion tube. The tubular member interacts with the actuation mechanism so that a movement of an actuation portion of the tubular member with respect to the base structure leads to the orientation of the orientable end.

The present invention concerns in general medical devices for use in endoscopies and laparoscopies and in particular a device for the manipulation of body tissue by endoscopy or laparoscopy.

Endoscopy is a mini-invasive procedure in which an inner part of the human or animal body is reached and viewed through natural body orifices or ducts, for example the esophagus or the rectum. This type of so-called endoluminal access allows the surgeon or doctor to see and/or treat inner portions of the orifice or body duct or tissues of internal organs accessible through them. Whereas the aforementioned interventions can also be carried out through conventional open surgery, endoscopy usually involves less pain, less risks and scars and faster recovery of the patient.

Endoscopy is typically carried out through an endoscope that comprises an insertion tube of small diameter with a distal end that is inserted into the orifice up to a desired internal position. Inside the insertion tube extend optical fibres that terminate in the distal end so as to allow an axial viewing from this distal end. The images of the internal position near to the distal end of the endoscope are transmitted to a video monitor that allows them to be seen by the surgeon. A control handle arranged at the proximal part of the endoscope allows the direction of the visual field and, in some cases, the actuation of suction, ventilation or rinsing devices possibly necessary for the endoscopy to be adjusted.

Since endoscopes can be used to carry out treatments in internal positions, some of them are equipped with channels through which it is possible to pass a tool or a surgical instrument. Normally, such channels extend along the length of the insertion tube up to its distal end with the result that the surgical instrument comes out and projects axially from the distal end. This limits the movement of the surgical instruments to axial and rotational movements around the orientation axis of the distal end of the endoscope, placing as many limits upon the complexity and variety of surgical or diagnostic procedures that can be carried out with this type of device.

Some of the aforementioned limits can be overcome through laparoscopy, in which access is obtained to the inside of the human or animal body through small incisions, carried out before the insertion of a laparoscope. The laparoscope comprises an insertion tube of small diameter with a distal end that is inserted into the incision up to a desired internal position. Inside the insertion tube optical fibres extend that terminate in the distal end so as to allow axial viewing from this distal end. The images of the internal position near to the distal end of the laparoscope are transmitted to a video monitor that allow them to be seen by the surgeon. Access through an incision is more direct, shorter and straighter than the way of access provided by the natural ducts of the body. This allows the use of laparoscopic insertion tubes that are shorter, rigider and straighter than endoscopic ones.

Laparoscopy, not being restricted to the presence and shape of the natural ducts of the body, allows the insertion of further surgical instruments through many separate incisions, the suitable positioning and orientation of which allows the positioning of the surgical instruments in various directions. Whilst avoiding the limitation of orientation and movement of the surgical instruments, this benefit can only be obtained at the expense of high invasiveness due to the large number of incisions. Indeed, it is essential to make access routes for the surgical instruments, which involves the use of “trocar” needles that require general anaesthetic, the risk of complications and infections and an increase in recovery time of the patient. The purpose of the present invention is therefore that of providing a device for carrying out the aforementioned mini-invasive procedures that allow greater freedom of orientation and positioning of the surgical instruments, without giving up the advantages typical of endoscopy, in other words that it is not very invasive at all and suitable for reaching places located deep inside the body.

A further purpose of the present invention is that of providing a device for the manipulation of tissue that is reliable, robust, easy to use and cost-effective, since it can be used with conventional endoscopes and laparoscopes without needing onerous adaptation measurements.

These and other purposes are accomplished through a device for manipulating body tissue comprising:
a base structure;
at least one tubular member having a proximal end and an orientable distal end that defines a seat for supporting a surgical instrument, said tubular member being connected to the base structure;
an actuation mechanism suitable for orientating said distal end of the tubular member to take it into certain operative configurations, said actuation mechanism being connected to the base structure;
means for a connection of the base structure to a distal end portion of an insertion tube of an endoscope or laparoscope, so that the actuation mechanism and the tubular member are arranged outside said insertion tube,
   in which said tubular member interacts with the actuation mechanism so that a movement of an actuation portion of the tubular member with respect to the base structure involves said orientation of the orientable end.

Thanks to the characteristics of the device according to the invention it is possible to obtain wide angle orientation of the surgical instruments with respect to the insertion tube, without interfering on the internal structure of the insertion tube, using a single endoscope or laparoscope and without needing further ways of access for the surgical instruments themselves.

Further conceptual developments of the invention and advantageous embodiments are the object of the dependent claims.

In the present invention it has been noted how particularly advantageous it is to provide a channel inside the tubular member that allows a surgical instrument to be transported from the proximal end of the tubular member up to its orientable distal end as well as allowing surgical instruments to be replaced during the intervention without having to remove and reinsert the insertion tube.

According to a further aspect of the present invention the tubular member comprises at least one transmission portion arranged between the actuation mechanism and the proximal end of the tubular member, in which this transmission portion forms a substantially rigid tubular rod. Thanks to the rigidity of the tubular rod it is possible to actuate the actuation mechanism without needing to foresee special guides to fix the tubular member to the insertion tube and, therefore, without needing to interfere with the typical structure of a conventional endoscope or laparoscope.

Alternatively, a guided transmission portion of the tubular member can be substantially flexible so as to be able to follow a deformed shape of a flexible insertion tube, for example of an endoscope.

According to a further aspect of the present invention, the actuation mechanism comprises an articulated frame distinct from the tubular member and connected with it. This advantageously allows the actuation functions of the actuation mechanism to be separated from the transportation and support functions of the surgical instruments of the tubular member.

According to a further aspect of the present invention, the articulated frame comprises a first end rotatably connected with a fixed hinge portion of the base structure and a second end slidably connected to a guide portion of the base structure more proximal with respect to the fixed hinge portion and the orientable distal end of the tubular member is connected to the articulated frame so that the deformation of the articulated frame results in the orientation of the orientable end. Thanks to this configuration of the movement restrictions of the actuation mechanism a particularly stable support of the orientable distal portion is obtained and an adjustment of the orientation of the surgical instruments does not lead to their simultaneous axial displacement.

So as to better understand the invention and appreciate its advantages some non-limiting example embodiments shall be described hereafter, with reference to the attached figures, in which:

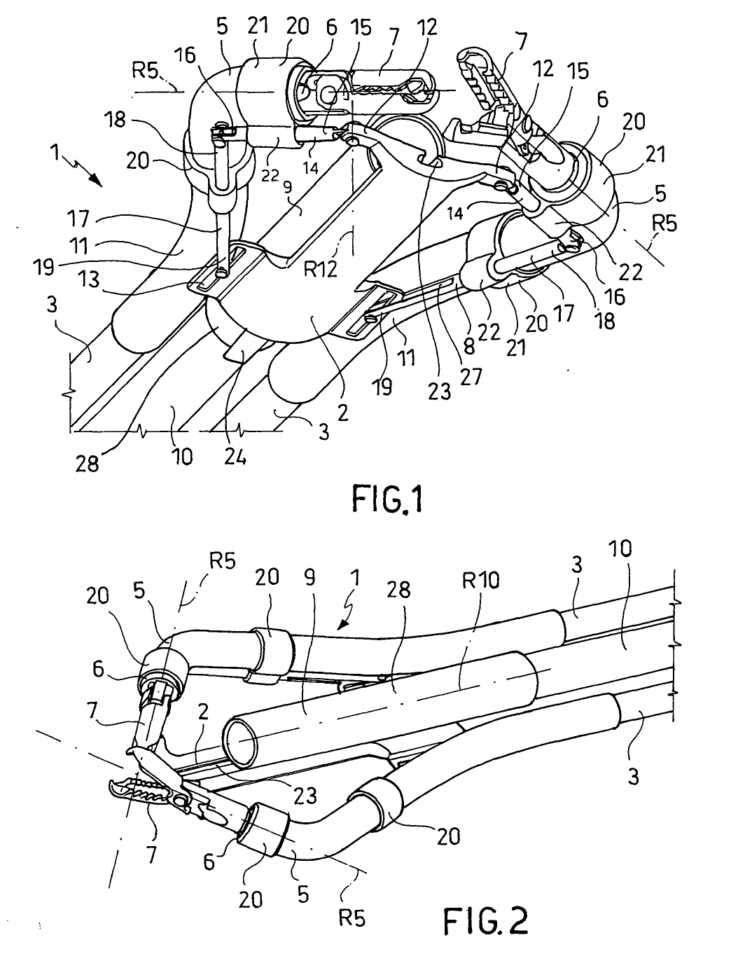

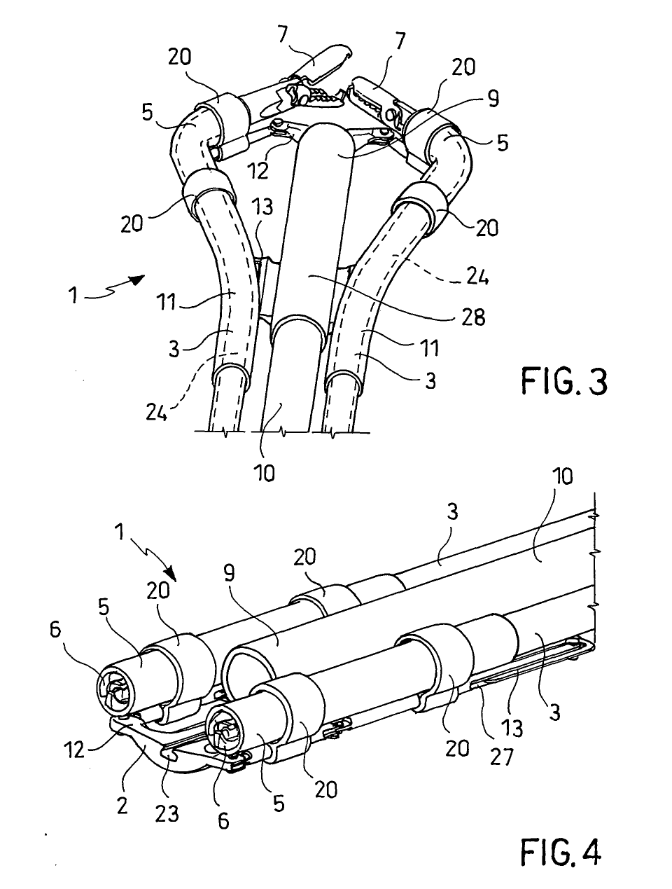

FIGS. 1, 2 and 3 are perspective views of the device according to an embodiment of the invention in an operative configuration mounted on the insertion tube of an endoscope or laparoscope;

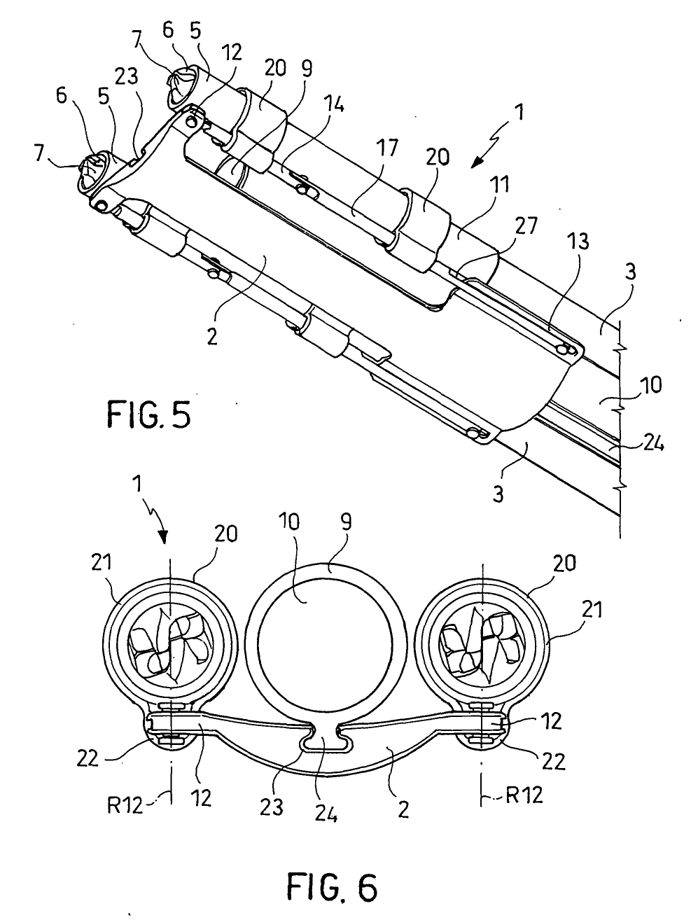

FIGS. 4 and 5 are perspective views of the device in FIG. 1 in a rest configuration;

FIG. 6 is a distal view of the device shown in FIGS. 4 and 5;

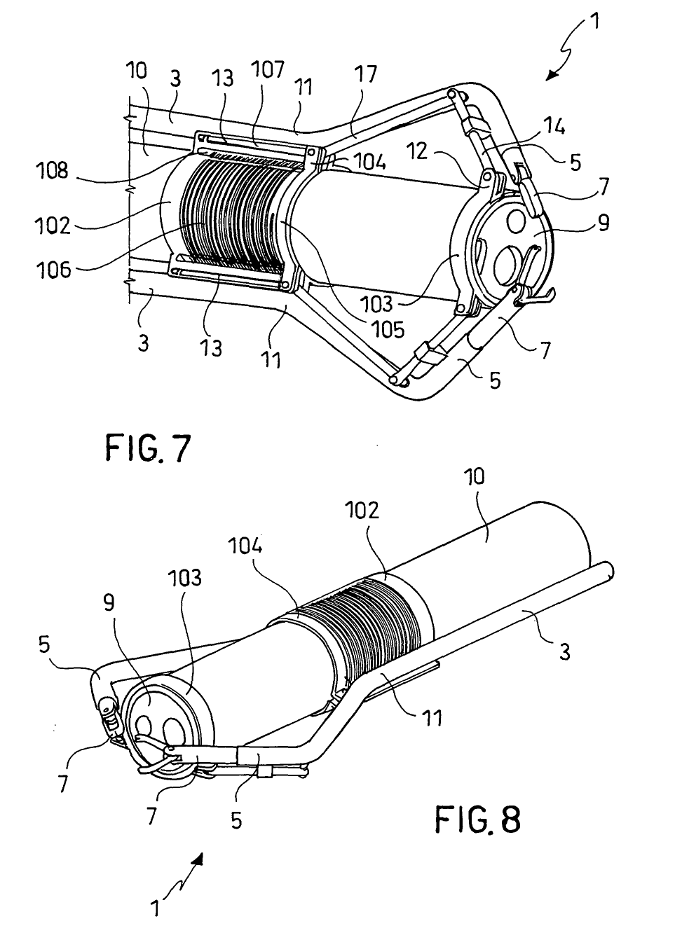

FIGS. 7 and 8 are perspective views of the device according to a further embodiment of the invention in an operative configuration mounted on the insertion tube of an endoscope or laparoscope;

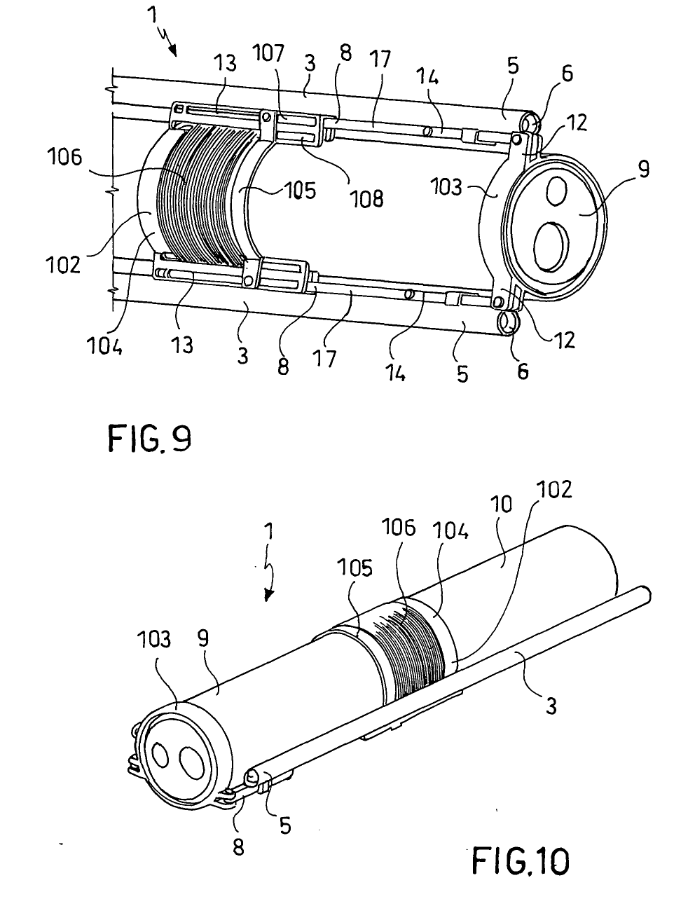

FIGS. 9 and 10 are perspective views of the device in FIG. 7 in a rest configuration;

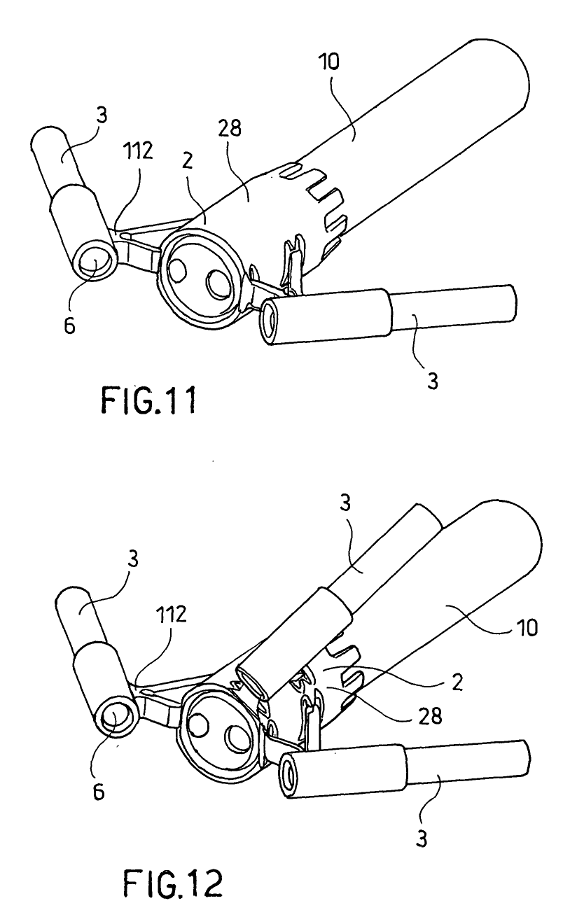

FIG. 11 is a perspective view of a detail of the device according to a further embodiment of the invention;

FIG. 12 is a perspective view of a detail of the device according to yet another embodiment of the invention;

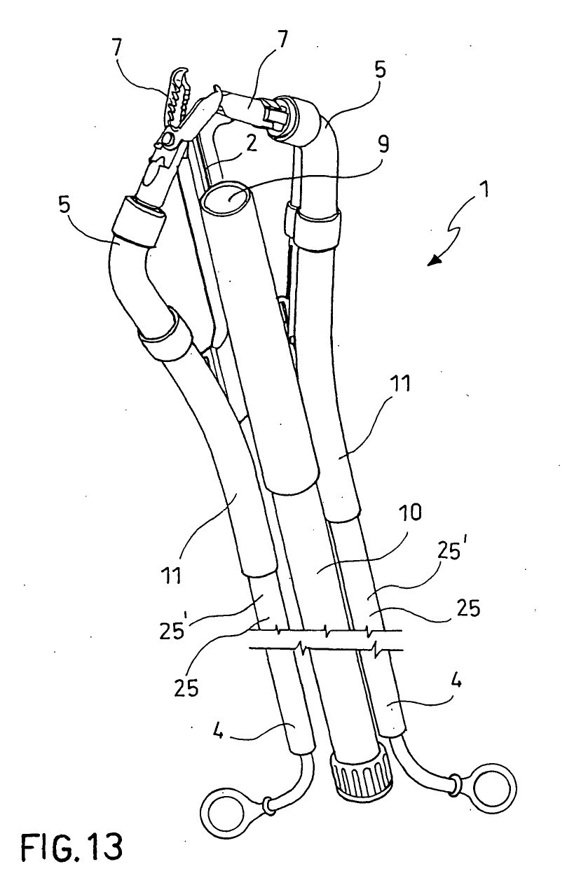

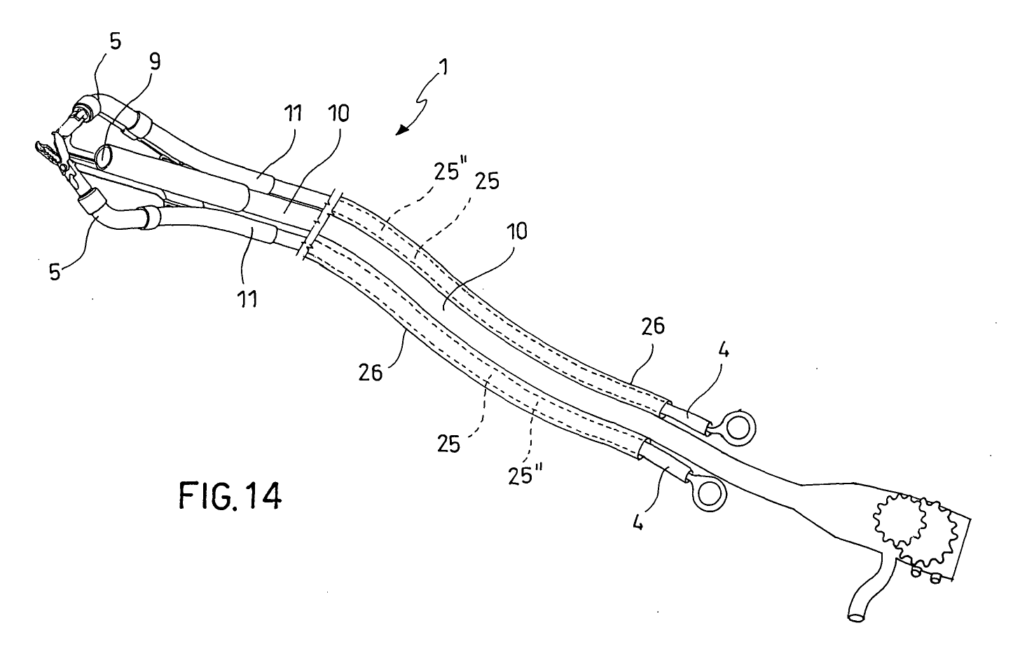

FIGS. 13 and 14 are schematised perspective views of the device according to two embodiments of the invention.

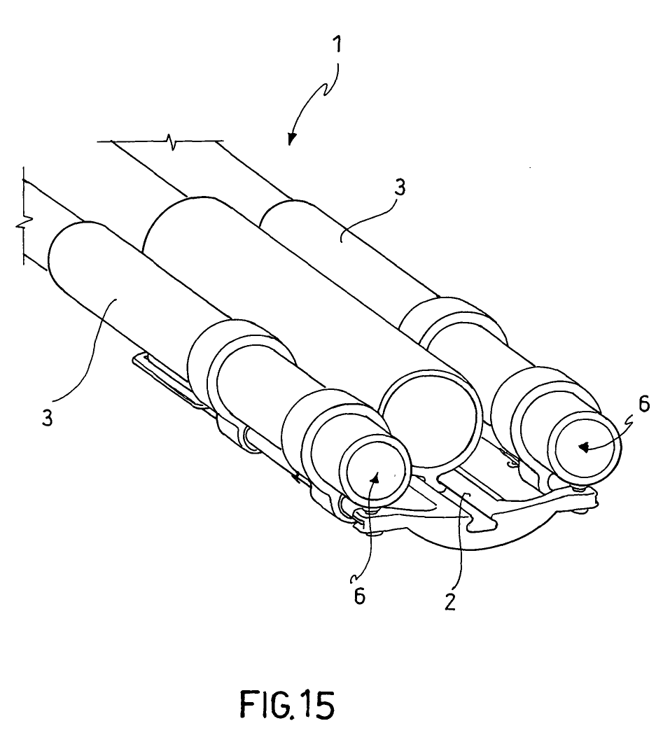

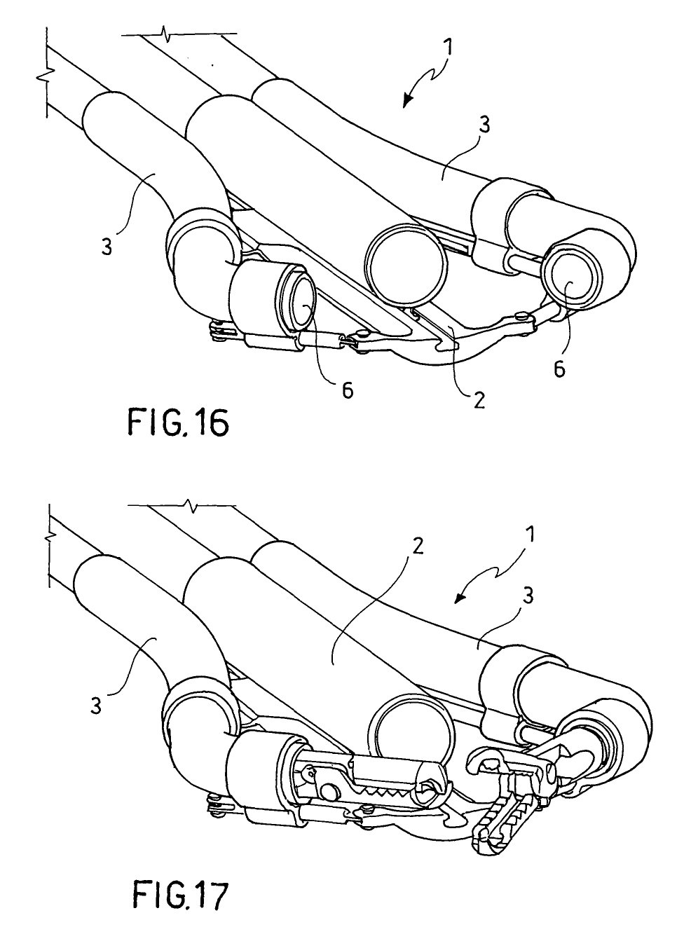

FIGS. 15 to 17 show the device according to an embodiment separate from the insertion tube of a laparoscope or endoscope.

With reference to FIG. 1, a device for manipulating body tissue is wholly indicated with reference numeral 1. The device 1 comprises a base structure 2 and at least one tubular member 3 with a proximal end 4 and an orientable distal end 5 that defines a seat 6 for supporting a surgical instrument, for example a grasper 7. The tubular member 3 is connected to the base structure 2 through an actuation mechanism, in particular an articulated frame 8. The actuation mechanism is suitable for orientating the distal end 5 of the tubular member 3 to take it into certain operative configurations, in particular to orientate it in inclined or transversal directions with respect to a longitudinal axis of the base structure 2 (that coincides with the longitudinal axis R10 of a distal end portion 9 of an insertion tube 10 that shall be described hereafter). The actuation mechanism 8 is also connected to the base structure 2.

The base structure 2 is in turn connected or able to be connected through suitable connection means to a distal end portion 9 of an insertion tube 10 of an endoscope or laparoscope, so that the actuation mechanism 8 and the tubular member 3 are arranged outside the insertion tube 10. The tubular member 3 is connected to the actuation mechanism 8 so that a movement of an actuation portion 11 of the tubular member 3 with respect to the base structure 2 leads to the aforementioned orientation of the orientable distal end 5.

As already stated previously, an embodiment foresees that the actuation mechanism comprises an articulated frame 8, preferably distinct from the tubular member 3 and connected with it through one or more connectors. This advantageously allows the functions of the two components of the device to be separated and, therefore, allows them to be better optimised both in terms of shape and in terms of the material.

In the embodiments shown in the figures, the device 1 comprises a plurality, and in particular two tubular members 3, each of which respectively interacts with an actuation mechanism 8.

In the following description reference shall be made to a single tubular member 3 and a single actuation mechanism, in other words a single articulated frame 8, but it should be understood that the characteristics of shape and functions as well as their interactions refer both to the embodiment with a single tubular member and to those with two or more tubular members and respectively two or more actuation mechanisms.

The articulated frame 8 comprises a first end rotatably connected with a fixed hinge portion 12 of the base structure 2 and a second end slidably connected, and in particular able to move longitudinally, to a guide portion 13 of the base structure 2 more proximal with respect to its fixed hinge portion 12. In this way, a movement of the second end of the articulated frame 8 along the guide 13 results in a deformation of the articulated frame 8 and, therefore, the orientation of the orientable end 5 connected with it.

According to an embodiment, the articulated frame 8 comprises a first distal shaft 14 having a distal end 15 rotatably connected with the fixed hinge portion 12 as well as a proximal end 16 rotatably connected with the distal end 18 of a second proximal shaft 17. A proximal end 19 of the second proximal shaft 17 is in turn connected, so that it can rotate and slide, with said axial guide portion 13 of the base structure 2, so as to form a triangular articulated frame.

At least the fixed hinge portion 12 defines a rotation axis R12 such that at least the distal shaft 14 can move substantially in only a single plane.

In the case of two opposite tubular members, the movements of the distal shafts of both of the articulated frames are preferably but not necessarily restricted to the same plane of movement.

According to an embodiment, a part from the fixed hinge portion 12, also the rotatable connection between the two distal and proximal shafts 14 and 16 and between the proximal shaft and the guide 13 are formed so as to limit the displacement and deformation movements of the entire articulated frame 8 substantially to a single plane. Regarding this, the proximal shaft preferably comprises a longitudinal slit 27 suitable for receiving the guide portion 13 so as to prevent transversal movements of the proximal shaft 17 with respect to the guide 13.

The hinge connections are preferably made through a pin that defines the rotation axis and the sliding guide 13 preferably comprises a rectilinear slot the direction of which defines the sliding direction and inside which the proximal end 19 of the proximal shaft 17 is fixed through a pin that defines the rotation axis R17 of the proximal end 19 of the proximal shaft 17 with respect to the guide 13. Advantageously, the proximal shaft 17 has a greater length than the length of the distal shaft 14. This allows a hyper-proportional relationship to be obtained between the actuation movement (along the guide 13) of the proximal shaft 17 and the angular orientation rotation of the distal shaft 14.

Advantageously, the orientable distal end 5 of the tubular member 3 is fixed to the distal shaft 14 of the articulated frame 8 so as to be orientated substantially in the same direction or in a direction parallel to the longitudinal extension of the distal shaft 14. This allows the bulk of the device 1 and of the entire endoscope or laparoscope to be limited as much as possible both during the insertion and removal from the patient's body and in the operative step at the location inside the body.

In order to allow optimal actuation of the articulated frame 8, as well as the connection of the orientable end 5 with the distal shaft 14, it is advantageously foreseen that a more proximal portion of the tubular member 3 of its orientable distal portion 5 is fixed to the proximal shaft 17 of the articulated frame 8, so that the movement of the tubular member 3 with respect to the base structure 2 directly translates into a sliding of the proximal shaft 17 along the guide portion 13 of the base structure 2.

According to an embodiment, rotatable connectors 20 are provided that connect the tubular member 3 with the articulated frame 8 and allow a rotation of the tubular member 3 around the longitudinal axes of the shafts 14, 17 of the articulated frame. In this way a further degree of freedom of movement or of adjustment of the position of the orientable distal ends 5 and, therefore, of the surgical instruments 7 one with respect to the other and with respect to the axial visual field of the endoscope or laparoscope is obtained.

Advantageously, the connectors 20 (both in the embodiment that allows the aforementioned further rotation, and in the embodiment in which this rotation is prevented) are double sleeve shaped defining a first tubular portion 21 that clutches said tubular member 3 completely surrounding it and a second tubular portion 22 that clutches the shaft 14, 17 of the frame 8, completely surrounding it. Alternatively, the two tubular portions can have an open clip-shaped profile.

In order to make the movement of the articulated frame 8 easier, advantageously, substantially in the entire area of the frame 8, the tubular member 3 is deformable.

In accordance with an embodiment, a first connector of the connectors 20 allows a relative longitudinal movement between said tubular member 3 and said articulated frame 8 and a second connector of the connectors 20 prevents such a relative longitudinal movement. This allows a movement of the articulated frame substantially free from restrictions due to incompatibility between the movements of the articulated frame and those of the tubular member.

Preferably, the first connector (fixed connection) connects the proximal shaft 17 with the tubular member 3 and the second connector (sliding connection) connects the distal shaft 14 with the tubular member 3, so as to avoid undesired longitudinal movements of the distal end of the tubular member with respect to the insertion tube.

A further degree of freedom of movement of the surgical instruments 7 with respect to the axial visual field of the insertion tube 10 can be obtained if the seat(s) 6 are made to support the surgical instruments so as to allow the rotation of the surgical instrument 7 around a longitudinal axis R5 of the orientable distal portion 5 of the tubular member 3.

According to an embodiment, the base structure 2 can be connected to the insertion tube 10 through a guide profile 23 that allows a connection in variable positions with a counter-guide profile 24 formed in the distal end portion 9 of the insertion tube 10. Preferably, the guide profile 23 and the counter-guide profile 24 define an adjustment direction substantially parallel to the longitudinal axis R10 of the distal end portion 9 of the insertion tube 10. Particularly advantageously, the guide profile 23 and the counter-guide profile 24 have substantially matching cross sections with an undercut, for example of the dovetailed type, to effectively prevent them from accidentally decoupling.

Advantageously, the coupling between the guide 23 and the counter-guide 24, is an interference coupling, for example of the press-fit type. Along the guide 23 and/or the counter-guide 24 elastically yielding knurled or toothed tracks can be foreseen to allow their mutual position to be adjusted by snap locking.

According to a preferred embodiment, the counter-guide profile 24 is formed on the outer surface of a preferably tubular connection portion 28 which can be connected, for example through press-fit with the distal end of the insertion tube 10. Of course, although the preferred connection has been described, the man skilled in the art, without departing from the present invention, can select similar embodiments that allow the base structure 2 to be connected to the insertion tube 10 in various positions (or, in other words, allowing the connection position to be adjusted) along the distal end portion 9 of the insertion tube 10.

According to the preferred embodiment, the base structure 2 is a body formed in a single piece, as shown for example in FIGS. 1 and 5.

According to an alternative embodiment (FIGS. 7 to 10), the base structure 102 comprises a distal portion 103 and a proximal portion 104 separate from the distal portion 103. The distal portion 103 forms the fixed hinge portion 12 and is connected to the distal end portion 9 of the insertion tube 10. The proximal portion 104 of the base structure forms the guide portion 13 and is also connected to the distal end portion 9 of the insertion tube 10, but in a distanced and proximal position with respect to the distal portion 103.

Advantageously, a sliding ring 105 is connected with the proximal end 19 of the proximal shaft 17 and slidably guided by the guide portion 13. A spring 106, preferably a helical extension spring or a similar elastic biasing member, acts between the proximal portion 104 and the sliding ring 105 in order to elastically bias the articulated frame 8 in its rest configuration. More advantageously, the spring 106 itself is guided and received either in the groove 107 of the guide portion 13 itself or in a separate parallel groove 108.

A similar or analogous elastic biasing member can also advantageously be provided in the other embodiments described and illustrated with the purpose of elastically biasing the actuation mechanism 8 permanently in the rest configuration. As an example, the aforementioned biasing member can be made through an elastic spring that acts between the base structure 2 and the articulated frame 8 or, alternatively, through an elastic material with spring-effect directly integrated in the tubular member close to the articulated frame 8.

The tubular member 3 defines a channel 24 on the inside that allows the surgical instrument 7 to be transported from the proximal end 4 of the tubular member up to its orientable distal end 5.

The tubular member 3 comprises at least one transmission portion 25 arranged between the actuation mechanism and the proximal end 4 of the tubular member. In accordance with an embodiment, the transmission portion 25 is formed from a substantially rigid tubular rod 25′. Thanks to the rigidity of the tubular rod 25′ it is possible to actuate the actuation mechanism 8 without needing to foresee special guides to fix the tubular member 3 to the insertion tube 10 and, therefore, without needing to interfere with the typical structure of a conventional endoscope or laparoscope. In accordance with an alternative embodiment, the transmission portion 25 comprises a substantially flexible tubular portion 25″ guided through a guide 26 that restricts its movement with respect to the insertion tube 10 of the endoscope so as to allow substantially only the movement in the longitudinal direction of the insertion tube 10. In the case of a flexible insertion tube, the guide 25, for example a deformable hose, connected and arranged parallel to the insertion tube 10, allows the flexible portion 25″, together with the insertion tube itself, to follow the irregular shape of a natural duct of the human or animal body.

The embodiments illustrated in FIGS. 11 and 12 provide that the function of the articulated frame be carried out thanks to the controlled deformability of the tubular member 3 itself that is rotatably connected to the base structure 2 in a single point. Such a rotatable connection can for example be obtained through an elastically deformable joint 112 without the use of a pin to define the rotation axis of the joint 112.

It is advantageously possible to manufacture and use the device described up to here as an accessory for existing endoscopes and laparoscopes, as well as to manufacture and use endoscopes or laparoscopes that comprise the device for the manipulation of body tissue as a removable or non-removable component.

Hereafter the operation of the device for the manipulation of body tissue according to the invention shall be described.

The device 1 is mounted on the insertion tube 10 of a laparoscope or endoscope through the coupling of the guide 23 of the base body 2 with the counter-guide 24 of the insertion tube, the mutual positioning in the desired assembly position and the locking of the connection through locking means, for example threaded means, snap means, friction means or other locking means.

By holding the proximal end 4 of the tubular member 3 pulled in the proximal direction, the proximal end 19 of the proximal shaft 17 of the articulated frame 8 is positioned in a proximal limit position in the guide 13 of the base structure 2, aligning both the articulated frame and the orientable distal end 5 of the tubular member 3 with the insertion tube. In this rest configuration, the bulk of the device 1 is minimal to allow the insertion of the laparoscope or endoscope in the patient's body up to the desired location.

After the positioning of the insertion tube 10, the device 1 is kept in the rest configuration and the surgical instrument 7 is passed through the channel 24 of the tubular member up to its distal end 5 where the surgical instrument 7 is received in the appropriate seat 6.

The orientation of the surgical instrument with respect to the axis R10 of the insertion tube is now easily possible through a movement in the distal direction of the actuation portion 11 of the tubular member 3 with respect to the insertion tube 10 (operative configuration of the device 1). Thanks to the channel 24 it is also possible to replace the surgical instrument during the intervention. To do so it is sufficient to pull the actuation portion 11 of the tubular member 3 in the proximal direction to take the device 1 into the rest configuration, in which the distal portion 5 of the tubular member 3 is substantially straight and allows the surgical instrument used up to here to be pulled out and replaced with a different instrument.

The removal of the endoscope or laparoscope from the patient's body takes place, as in the case of insertion, with the device 1 in rest configuration.

The device according to the present invention has numerous advantages.

Thanks to the characteristics of the device according to the invention it is possible to obtain wide angles of the surgical instruments with respect to the insertion tube, using a single endoscope or laparoscope, without interfering on the internal structure of the insertion tube and without needing further points of access (incisions) for the surgical instruments themselves.

The device 1 allows a surgical instrument to be transported from the proximal end of the tubular member up to its orientable distal end as well as allowing the surgical instruments to be replaced during the intervention without having to remove and reinsert the insertion tube itself.

In the embodiment with a rigid transmission rod it is possible to actuate the actuation mechanism without needing to foresee special guides to fix the tubular member to the insertion tube and, therefore, without needing to interfere with the typical structure of a conventional endoscope or laparoscope.

In the embodiment with a flexible transmission portion it is possible to follow a deformed shape of a flexible insertion tube, for example of an endoscope.

Thanks to the particular shape of the articulated frame and to the fact that it is distinct from the tubular member 3, an advantageous separation of the orientation function from the transportation, support and actuation functions of the surgical instruments is obtained.

Thanks to the particular configuration of the articulated frame and, therefore, to the restrictions of movement of the actuation mechanism, a particularly stable and controllable movement of the surgical instruments and an adjustment of the orientation of the surgical instruments that does not lead to their simultaneous axial movement is obtained.

1. Device (1) for manipulating body tissue comprising:
a base structure (2, 102);
at least one tubular member (3) having a proximal end (4) and an orientable distal end (5) that defines a seat (6) for supporting a surgical instrument (7), said tubular member (3) being connected to the base structure (2);
an actuation mechanism (8) suitable for orientating said distal end (5) of the tubular member (3) to take it into certain operative configurations, said actuation mechanism (8) being connected to the base structure (2);
means (23, 24) for the connection of the base structure (2) to a distal end portion (9) of an insertion tube (10) of an endoscope or laparoscope, so that the actuation mechanism (8) and the tubular member (3) are arranged outside of said insertion tube (10),
in which said tubular member (3) interacts with the actuation mechanism (8) so that a movement of an actuation portion (11) of the tubular member (3) with respect to the base structure (2) leads to said orientation of the orientable end (5).

2. Device (1) according to claim 1, in which said tubular member (3) internally defines a channel (24) that allows said surgical instrument (7) to be transported from said proximal end (4) of the tubular member (3) up to its orientable distal end (5).

3. Device (1) according to any one of the previous claims, in which said tubular member (3) comprises at least one transmission portion (25) arranged between the actuation mechanism (8) and the proximal end (4) of the tubular member (3), said transmission portion (25) comprising a substantially rigid tubular rod (25′).

4. Device (1) according to any one of the previous claims, in which said tubular member (3) comprises:
at least one guided transmission portion (25), arranged between the actuation mechanism (8) and the proximal end (4) of the tubular member (3);
a guide (26) suitable for restricting the movement of the transmission portion (25) with respect to the insertion tube (10) so as to allow the movement of the guided transmission portion (25) substantially only in the longitudinal direction of the insertion tube (10).

5. Device (1) according to the previous claim, in which said guided transmission portion (25) comprises a flexible portion (25″) so as to be able to follow a deformed shape of a flexible insertion tube.

6. Device (1) according to any one of the previous claims, in which said actuation mechanism comprises an articulated frame (8).

7. Device (1) according to the previous claim, in which said articulated frame (8) comprises a first end (15) rotatably connected with a fixed hinge portion (12) of the base structure (2) and a second end (19) slidably connected with a guide portion (13) of the base structure (2) more proximal with respect to said fixed hinge portion (12), so that a movement of the second end (19) of the articulated frame (8) along said guide portion (13) leads to a deformation of the articulated frame (8),
in which said orientable distal end (5) of the tubular member (3) is connected to said articulated frame (8) so that said deformation of the articulated frame (8) leads to said orientation of the orientable end (5).

8. Device (1) according to the previous claim, in which said articulated frame (8) comprises
a distal shaft (14) having a distal end (15) rotatably connected with said fixed hinge portion (13) as well as a proximal end (16);
a proximal shaft (17) having a proximal end (19) slidably connected with said guide portion (13) and a distal end (18) rotatably connected with the proximal end (16) of the distal shaft (14), so as to form a triangular shaped articulated frame.

9. Device (1) according to the previous claim, in which at least the fixed hinge portion (12) defines a rotation axis (R12) such that said distal shaft (14) can move substantially in only a single plane.

10. Device (1) according to claim 8 or 9, in which said proximal shaft (17) has a greater length than the length of the distal shaft (14).

11. Device (1) according to any one of claims 8 to 10, in which said orientable distal portion (5) of the tubular member (3) is fixed to said distal shaft (14) of the articulated frame (8) so as to be orientated substantially in the same direction in which said distal shaft (14) is orientated.

12. Device (1) according to any one of claims 8 to 11, in which a portion of the more proximal tubular member (3) of its orientable distal end (5) is fixed to said proximal shaft (17) of the articulated frame (8), so that said movement of the tubular member (3) with respect to the base structure (2) leads to the sliding of the proximal shaft (17) in the guide portion (13) of the base structure (2).

13. Device (1) according to any one of claims 6 to 12, comprising rotatable connection means (20, 21, 22) that connect said tubular member (3) with said articulated frame (8) so that the position of the tubular member (3) can be adjusted through rotation of the tubular member (3) around the longitudinal axes of the shafts (14, 17) of the articulated frame (8).

14. Device (1) according to any one of claims 8 to 13, comprising one or more double-sleeve connectors (20) forming a first tubular portion (21) that clutches said tubular member (3) completely surrounding it and a second tubular portion (22) that clutches a shaft (14, 17) of said articulated frame (8) completely surrounding it.

15. Device (1) according to claim 14, in which a first connector of the connectors (20) allows a relative longitudinal movement between said tubular member (3) and said articulated frame (8) and a second connector of the connectors (20) prevents such a relative longitudinal movement.

16. Device (1) according to claim 15, in which said first connector connects the proximal shaft (17) with the tubular member (3) and said second connector connects the distal shaft (14) with the tubular member (3).

17. Device (1) according to any one of claims 6 to 16, in which, in the area of the articulated frame (8), said tubular member (3) is deformable.

18. Device (1) according to any one of the previous claims, in which said seat (6) for supporting a surgical instrument (7) allows the rotation of the surgical instrument (7) around a longitudinal axis (R5) of said orientable distal portion (5) of the tubular member (3).

19. Device (1) according to any one of the previous claims, comprising adjustment means (23, 24) that allow the base structure (2) to be connected to the insertion tube (10) in various positions along said distal end portion (9) of the insertion tube (10).

20. Device (1) according to any one of the previous claims, in which said base structure (2) defines a guide profile (23) having a shape such as to be able to be coupled in various positions with a counter-guide profile (24) connected with the distal end portion (9) of the insertion tube (10), in which said guide profile (23) and said counter-guide profile (24) define an adjustment direction substantially parallel to a longitudinal axis (R10) of the distal end portion (9) of the insertion tube (10).

21. Device (1) according to the previous claim, in which the counter-guide profile (24) is formed on the outer surface of a connection portion (28) that can be connected with the distal end of the insertion tube (10).

22. Device (1) according to the previous claim, in which said connection portion (28) can be connected with the distal end of the insertion tube (10) through press-fit.

23. Device (1) according to any one of the previous claims, comprising biasing means suitable for elastically biasing the actuation mechanism (8) in the rest configuration.

24. Device (1) according to the previous claim, in which said biasing means comprise an elastic spring that acts between the base structure (2) and said articulated frame (8).

25. Device (1) according to the previous claim, in which said elastic spring is preloaded in traction.

26. Device (1) according to claim 23, in which said biasing means are integrated in said tubular member near to the articulated frame (8).

27. Device (1) according to any one of the previous claims, in which said base structure (2) is a body formed in a single piece.

28. Device (1) according to any one of claims 1 to 26, in which said base structure (102) comprises:
a distal portion (103) that can be fixed to said distal end portion (9) of the insertion tube (10) and comprising said fixed hinge portion (12);
a proximal portion (104) separate from the distal portion (103) and able to be fixed to said distal end portion (9) of the insertion tube (10) in a distanced and proximal position with respect to the distal portion (103), said proximal portion (104) comprising said guide portion (13).

29. Device (1) according to claim 28, comprising a sliding ring connected with the proximal end (19) of the proximal shaft (17) and slidably guided by said guide portion (13), in which said biasing means act between said proximal portion (104) and said sliding ring.

30. Device (1) according to any one of the previous claims, in which said actuation mechanism comprises an articulated frame (8) distinct from the tubular member (3) and connected with it.

31. Device (1) according to any one of the previous claims, comprising two or more of said tubular members (3) and two or more of said actuation mechanisms (8), in which each tubular member (3) respectively interacts with one of the actuation mechanisms (8) in the way claimed in one or more of the previous claims.

32. Endoscope comprising a device (1) according to any one of the previous claims.

33. Laparoscope comprising a device (1) according to any one of claims 1 to 31.
